(19) United States
(12) Patent Application Publication
(10) Pub. No.: US 2015/0094664 A1
(43) Pub. Date: Apr. 2, 2015

(54) DRUG DELIVERY DEVICE

(71) Applicant: SYNDEO LLC, Tokyo (JP)

(72) Inventor: Masataka MIZUKOSHI, Tokyo (JP)

(73) Assignee: SYNDEO LLC, Tokyo (JP)

(21) Appl. No.: 14/499,968

(22) Filed: Sep. 29, 2014

(30) Foreign Application Priority Data
Oct. 1, 2013 (JP) 2013-206673
Jul. 15, 2014 (JP) 2014-145447

(51) Int. Cl. A61M 5/142

(52) U.S. Cl. CPC A61M 5/142; A61M 2005/1585
USPC 604/150

(57) ABSTRACT

A drug delivery device includes a cylindrical casing which has two openings on opposite sides, a lid which has an air inflow hole and is joined at one end of the cylindrical casing so as to close one of the openings. An internal space, in which an inflating agent and a drug are sealed while being separated from each other, is formed by at least the cylindrical casing and the lid, the internal space communicates with the air inflow hole and a delivery hole through which the drug is delivered, the inflating agent is formed of a material which is mainly composed of iron, iron oxide is produced by at least oxygen in the inflow air, which causes the inflating agent to be inflated, and as the inflating agent is inflated, the drug is delivered through the delivery hole.

### TECHNICAL FIELD

The present invention relates to the technical field of a drug delivery device which delivers a drug, sealed in the internal space, through a delivery hole.

### BACKGROUND ART

There is a method in which a needle is stuck into the muscle or the vein to administer a drug to a patient by means of injection. In this method, sending a drug into the body over a prolonged time requires the use of an infusion stand for supporting the drug or of a long tube for sending the drug from a reservoir.

For example, a needle is stuck into the skin and a liquid drug is supplied for injection from a reservoir by means of electrophoresis, a pump, a motor, or the like.

### SUMMARY OF INVENTION

### Problems To Be Solved by the Invention

However, these methods require a reservoir as well as an electric circuit and a pump, which makes the device used massive and complex in structure, so that it is inconvenient to use especially in daily life.

A drug delivery device of the present invention is intended to overcome the above-described problems and realize simplification of the structure and reduction of the size.

### Solution to Problem

First, a drug delivery device according to the present invention includes a cylindrical casing and a lid. The cylindrical casing is formed in a shape which is at least partly cylindrical and has two openings on opposite sides. The lid has an air inflow hole and is joined at one end of the cylindrical casing so as to close one of the openings. An internal space, in which an inflating agent and a drug are sealed while being separated from each other, is formed by at least the cylindrical casing and the lid, the internal space communicates with the air inflow hole and a delivery hole through which the drug is delivered, the inflating agent is formed of a material which is mainly composed of iron, and upon inflow of air through the air inflow hole, iron oxide is produced by at least oxygen in the inflow air, which causes the inflating agent to be inflated, and as the inflating agent is inflated, pressure is applied to the drug and the drug is delivered through the delivery hole.

Thus, as the inflating agent is inflated, the drug sealed in the internal space is delivered through the delivery hole.

Second, according to the drug delivery device of the present invention, a sealing film, which closes the air inflow hole, is attached on the lid to stop delivery of the drug through the delivery hole.

Thus, the inflow of air to the internal space through the air inflow hole is stopped by the sealing film.

Third, according to the drug delivery device of the present invention, the drug is sealed in the internal space while being covered with a barrier film, and the barrier film is entirely positioned between the drug and the cylindrical casing and between the drug and the inflating agent.

Thus, the drug is separated from the inflating agent, and a non-biocompatible component such as a metal ion is unlikely to enter into the drug from the inflating agent and the cylindrical casing.

Fourth, according to the drug delivery device of the present invention, a backflow prevention film, which prevents inflow of the drug to the inflating agent, is disposed at least between the inflating agent and the drug in the internal space.

Thus, the moisture of the drug is unlikely to flow into the inflating agent.

Fifth, according to the drug delivery device of the present invention, the cylindrical casing is provided with a cylindrical end having the delivery hole, and a needle which is stuck into skin or a tube with the needle joined at a tip portion is mounted at the cylindrical end.

Thus, the drug delivered through the delivery hole is injected into the skin from the needle.

### Advantageous Effects of Invention

According to the present invention, the drug sealed in the internal space is delivered through the delivery hole as the inflating agent is inflated. Thus, the need for structures such as a reservoir, an electric circuit, and a pump, is eliminated, and simplification of the structure as well as reduction of the size of the device can be realized.

### BRIEF DESCRIPTION OF THE DRAWINGS

FIG. 1, showing a first embodiment of a drug delivery device of the present invention along with FIG. 2 through FIG. 8, is a cross-sectional view showing the overall structure of the drug delivery device;

FIG. 2, showing the assembly procedure of the drug delivery device along with FIG. 3 and FIG. 4, is a cross-sectional view showing a state where a drug covered with a barrier film is inserted inside a cylindrical casing;

FIG. 3 is a cross-sectional view showing a state where a backflow prevention film and an inflating agent covered with an air shutoff film are sequentially inserted inside the cylindrical casing;

FIG. 4 is a cross-sectional view showing a state where a lid is joined on the cylindrical casing;

FIG. 5, showing the work and the operation during use of the drug delivery device along with FIG. 6 and FIG. 7, is a cross-sectional view showing a state where the cylindrical casing is fixed on the skin with an adhesive tape and a needle is stuck into the skin;

FIG. 6 is a cross-sectional view showing a state where a sealing film is detached from the lid, a boring jig is inserted into an air inflow hole, and a through-hole is formed in the air shutoff film;

FIG. 7 is a cross-sectional view showing a state where air flows to the inside of the air shutoff film, the inflating agent is inflated to deliver the drug through a delivery hole, and the drug is injected from the needle into the body;

FIG. 8 is a cross-sectional view showing one example of the state of use where the needle is mounted at a cylindrical end;

FIG. 9, showing a second embodiment of the drug delivery device of the present invention along with FIG. 10 through FIG. 19, is a cross-sectional view showing the overall structure of the drug delivery device;

FIG. 10, showing the assembly procedure of the drug delivery device along with FIG. 11 through FIG. 14, is a cross-sectional view showing a state where a needle-like structure is joined on the cylindrical casing;

FIG. 11 is a cross-sectional view showing a state where the drug covered with the barrier film and the inflating agent held inside the air shutoff film are sequentially inserted inside the cylindrical casing;

FIG. 12 is a cross-sectional view showing a state where the lid is joined on the cylindrical casing;

FIG. 13 is a cross-sectional view showing a state where the sealing film is attached on the lid and the air inflow hole is closed;

FIG. 14 is a cross-sectional view showing a state where an attaching tape is attached on a part of the drug delivery device and a syringe is formed;

FIG. 15, showing the work and the operation during use of the drug delivery device along with FIG. 16 and FIG. 17, is a cross-sectional view showing a state where the adhesive tape is attached on the skin and needle parts are stuck into the skin;

FIG. 16 is a cross-sectional view showing a state where the sealing film is detached from the lid, the boring jig is inserted into the air inflow hole, and the through-hole is formed in the air shutoff film;

FIG. 17 is a cross-sectional view showing a state where air flows to the inside of the air shutoff film and the inflating agent is inflated to deliver the drug through the delivery hole;

FIG. 18 is a cross-sectional view showing a first modified example of the drug delivery device; and

FIG. 19 is a cross-sectional view showing a second modified example of the drug delivery device.

### DETAILED DESCRIPTION OF EMBODIMENTS

### First Embodiment

In the following, a first embodiment of a drug delivery device of the present invention will be described with reference to the accompanying drawings.

In the drug delivery device according to the first embodiment, a delivery hole is formed at its cylindrical end; in the following description, the direction in which this delivery hole extends is defined as the vertical direction, and the direction toward the tip of the cylindrical end is defined as the lower side to denote the vertical direction. However, the vertical direction denoted hereinafter is intended for the purpose of illustration, and the implementation of the present invention shall not be limited by such direction.

### (The Overall Structure of the Drug Delivery Device According to the First Embodiment)

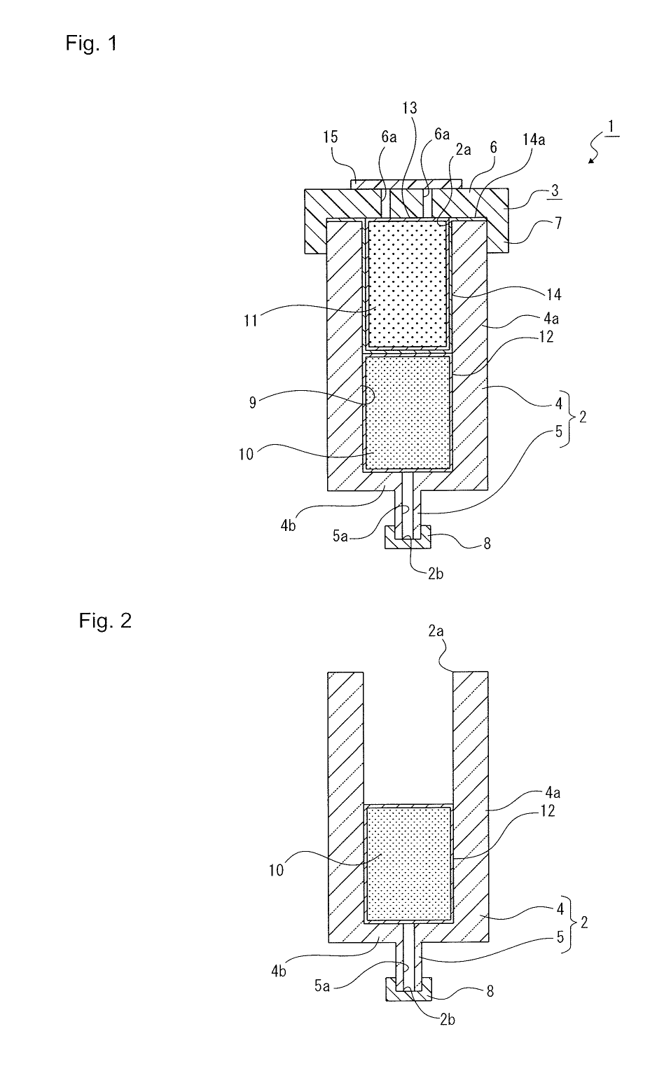

First, the overall structure of a drug delivery device 1 according to the first embodiment will be described (see FIG. 1).

The drug delivery device 1 has a cylindrical casing 2 formed in a cylindrical shape penetrated in the vertical direction, and a lid 3 which is joined on the upper end of the cylindrical casing 2.

The cylindrical casing 2 is, for example, integrally formed of a main body 4 and a cylindrical end 5 made of a highly rigid transparent or translucent material.

The main body 4 is constituted of a cylindrical part 4a, which is formed in a circular cylindrical shape or a rectangular cylindrical shape, and a coupling part 4b, which has a shape of an inner flange projecting inward from the lower end of the cylindrical part 4a. The cylindrical end 5 is formed in a circular cylindrical shape or a rectangular cylindrical shape so as to protrude downward from an inner circumferential portion of the coupling part 4b. The space inside the cylindrical end 5 is formed as a delivery hole 5a.

In the above-described example, the parts of the cylindrical casing 2 are integrally formed; however, at least one of the cylindrical part 4a, the coupling part 4b, and the cylindrical end 5 may be formed separately from the others, and these separately formed members may be joined together to constitute the cylindrical casing 2. Moreover, the cylindrical casing 2 is not required to be entirely formed of a transparent or translucent material, as long as at least the portion of the cylindrical part 4a where a drug to be described later is sealed is formed of a transparent or translucent material. The cylindrical casing 2 being transparent or translucent allows the degree of consumption of a drug sealed in the cylindrical casing 2 to be determined with the naked eyes.

Where the cylindrical casing 2 has an outer diameter (outer shape) of for example, approximately 10 mm, its thickness is 0.5 mm to 2.0 mm and its length is 50 mm to 100 mm, for example.

The lid 3 is formed of a metal material or a resin material, and is constituted of a closing part 6, which closes an opening 2a on the upper side of the cylindrical casing 2, and a cover part 7, which covers the upper end of the cylindrical casing 2 from the outer circumferential side. The closing part 6 is formed in a plate-like shape facing the vertical direction, and the cover part 7 protrudes downward from an outer circumferential portion of the closing part 6.

An air inflow hole 6a is formed in the closing part 6 so as to penetrate the closing part 6 in the vertical direction. Multiple air inflow holes 6a may be formed, and in the example shown in FIG. 1, etc., multiple air inflow holes 6a, 6a, . . . are formed.

The lid 3 has the cover part 7 joined on the cylindrical casing 2 by means such as screws, bonding, or thermocompression bonding, while its closing part 6 covers the upper surface of the cylindrical part 4a. When the lid 3 is joined on the cylindrical casing 2, the opening 2a on the upper side of the cylindrical casing 2 is closed by the closing part 6.

If the drug delivery device 1 is a type which is repeatedly usable, it is necessary to repeatedly seal a drug and an inflating agent, to be described later, inside the drug delivery device 1. It is therefore desirable that the lid 3 is joined on the cylindrical casing 2 with removable screws, etc., without being joined on the cylindrical casing 2 by means of bonding, thermocompression bonding, or the like.

A cap 8 is mounted at the tip portion of the cylindrical end 5. The cap 8 is detachable from the cylindrical end 5, and is formed of a metal material or a resin material. When the cap 8 is mounted at the cylindrical end 5, an opening 2b on the lower side of the cylindrical casing 2, namely, the opening at the tip of the cylindrical end 5 is closed by the cap 8. Thus, entry of foreign substances such as moisture and dust through the delivery hole 5a into the cylindrical casing 2 is prevented by the cap 8.

When the lid 3 is joined on the cylindrical casing 2 as described above, an internal space 9 is formed in the cylindrical part 4a. The internal space 9 communicates with the delivery hole 5a of the cylindrical end 5 and the air inflow holes 6a of the lid 3.

In the internal space 9, a drug 10 and an inflating agent 11 are sealed while being separated, for example, in the vertical direction.

The drug 10 is positioned under the inflating agent 11. The drug 10 is a water-soluble drug.

Examples of the drug 10 include insulin agent, opioid analgesic, antipyretic analgesic, steroidal anti-inflammatory agent, vasodilating drug, antiarrhythmic agent, hypotensive drug, local anesthetic, hormonal agent, antihistamine agent, general anesthetic, hypnotic analgesic agent, antiepileptic agent, psychoneurotic agent, skeletal muscle relaxant, autonomic agent, antiparkinsonian agent, diuretic, vasoconstrictive, and respiratory stimulant.

The drug 10 is generally a liquid. However, as will be described later, the drug 10 may be turned into a gel with low-molecular collagen or gelatin so that, when a needle or a tube with a needle joined at the tip portion is mounted at the cylindrical end 5 and the needle is stuck into the skin while the cylindrical casing 2 is fixed on the skin, the drug 10 sealed in the internal space 9 is dissolved by the body heat. The drug 10 is covered with the barrier film 12, and is in contact with all the surrounding members through the barrier film 12. Thus, the barrier film 12 is entirely positioned between the drug 10 and the cylindrical casing 2 and between the drug 10 and the inflating agent 11.

Before the delivery of the drug 10 is started, an insertion hole to be described later is formed in the barrier film 12 by inserting a puncture jig (not shown) through the delivery hole 5a of the cylindrical end 5, and after a needle or a tube with a needle joined at the tip portion is mounted at the cylindrical end 5, the drug delivery device 1 is used by inflating the inflating agent 11.

The barrier film 12 is formed of, for example, ethylene-vinyl acetate copolymer, polyethylene, polyethylene and ethylene-vinyl acetate copolymer, cyclic olefin copolymer, or polypropylene.

As described above, since the barrier film 12 is entirely in contact with all the surrounding members, the drug 10 covered with the barrier film 12 is reliably separated from the inflating agent 11, and entry of a non-biocompatible component such as a metal ion into the drug 10 from the inflating agent 11 and the cylindrical casing 2 can be prevented, so that entry of a non-biocompatible component into the body can be prevented.

The inflating agent 11 is formed of a material mainly composed of metal powder such as iron powder, and is inflated as oxygen and moisture in the air produce iron oxide. More particularly, the inflating agent 11 is mainly composed of metal powder such as iron powder, and is formed of a metal halide such as salt or a reaction accelerator such as metal sulfate, water, and a water retention agent such as a polymer absorption agent.

The inflating agent 11 is sealed in the internal space 9 while being held, for example, inside the air shutoff film 13 and shutoff from air. The air shutoff film 13 is formed of, for example, an ethylene-vinyl alcohol copolymer resin or a nylon-based (synthetic polymeric polyamide-based) synthetic resin.

The air shutoff film 13 is entirely held in close contact with the inner surface of the backflow prevention film to be described later and the lower surface of the lid 3. However, the backflow prevention film is not always used; in such a case, the air shutoff film 13 is entirely held in close contact with the inner circumferential surface of the cylindrical casing 2, the lower surface of the lid 3, and the upper surface of the barrier film 12.

When a through-hole to be described later is formed in the air shutoff film 13, the air flowing in through the air inflow holes 6a of the lid 3 turns the iron into iron oxide, which causes the inflating agent 11 to generate heat and to be inflated to about double its initial volume. As the inflating agent 11 is inflated, pressure is applied to the drug 10 by the inflation, causing the drug 10 to be delivered through the delivery hole 5a. Since the inflating agent 11 is inflated to about double its initial volume, for example, the inflating agent 11 having a thickness of 10 mm can deliver the drug 10 having a thickness of 10 mm through the delivery hole 5a.

As described above, since the inflating agent 11 is covered with the air shutoff film 13, the inflating agent 11 is shut off from the air. It is therefore unlikely that the inflating agent 11 is accidentally inflated before delivery of the drug 10, and accidental delivery of the drug 10 can be prevented. Moreover, since the inflating agent 11 can be stored before being inflated while its volume is small, the size of the internal space 9 can be made smaller by the saved volume and the size of the drug delivery device 1 can be reduced.

For example, where it is possible to sufficiently shut off the air from the inflating agent 11 using the cylindrical part 4a, the lid 3, and the sealing film to be described later, it is not absolutely necessary that the inflating agent 11 is sealed inside the air shutoff film 13, and instead, for example, the inflating agent 11 may be sealed in the internal space 9 while being held in a container bag formed of an air-permeable non-woven fabric, etc.

In the internal space 9, the backflow prevention film 14 except for one part is disposed. The backflow prevention film 14 is formed of, for example, ethylene-vinyl acetate copolymer, polyethylene, polyethylene and ethylene-vinyl acetate copolymer, cyclic olefin copolymer, or polypropylene.

The backflow prevention film 14 is, while being covered by the air shutoff film 13, disposed so as to cover the inflating agent 11 from the sides of the outer surfaces except for the upper side, and a part of the upper end side of the backflow prevention film 14 is provided as a pressed part 14a which projects outward. The backflow prevention film 14 is mounted on the cylindrical casing 2 by having the pressed part 14a placed on the upper surface of the cylindrical part 4a of the cylindrical casing 2 and the pressed part 14a pressed from above by the closing part 6 of the lid 3.

When the backflow prevention film 14 is thus used, even if the drug 10 should flow to the inflating agent 11 side along the wall surface of the cylindrical part 4a, the moisture contained in the drug 10 can be prevented by the backflow prevention film 14 from flowing into the inflating agent 11, so that the proper function of the inflating agent 11 can be secured.

A sealing film 15 is attached on the upper surface of the lid 3. As the sealing film 15, an adhesive tape, etc. which can be repeatedly attached to and detached from the lid 3 is used. The sealing film 15 seals the air inflow holes 6a of the lid 3.

When the drug delivery device 1 is used (when the drug is administered), the sealing film 15 is detached from the lid 3, and as the lid 3 is detached, the air inflow holes 6a are released from the sealed state.

With the sealing film 15 being thus attached on the lid 3, the air inflow holes 6a are sealed when the drug delivery device 1 is not in use, so that it is unlikely that a through-hole is formed in the air shutoff film 13 by a foreign substance accidentally entering the air inflow hole 6a while the drug delivery device 1 is not in use, and accidental use of the drug delivery device 1 can be prevented.

In addition, with the sealing film 15 being attached on the lid 3, it is unlikely that the airflow into the air inflow hole 6a is inhibited due to clogging caused by a foreign substance entering the air inflow hole 6a, and the drug 10 can be reliably delivered to the outside when the drug delivery device 1 is used.

### (The Assembly Procedure of the Drug Delivery Device According to the First Embodiment)

Next, the assembly procedure of the above-described drug delivery device 1 will be described (see FIG. 1 through FIG. 4).

When the cylindrical casing 2 is not integrally formed but is constituted of multiple separate members, these separately formed members are joined to form the cylindrical casing 2.

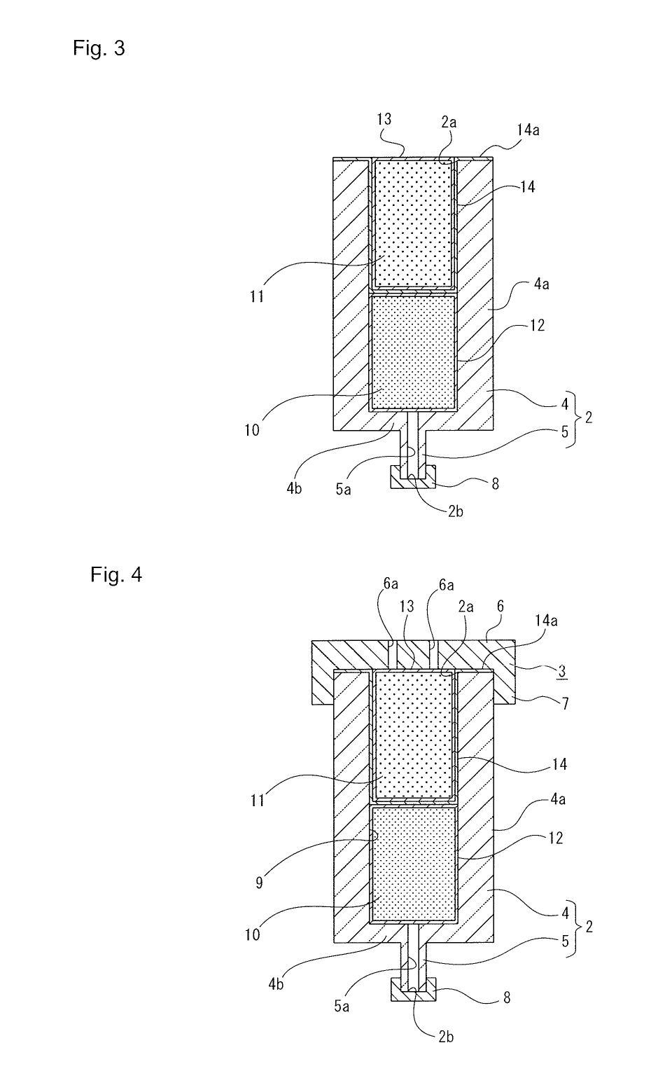

First, the drug 10 covered with the barrier film 12 is inserted into the cylindrical casing 2, and the cap 8 is mounted at the cylindrical end 5 (see FIG. 2). Next, the backflow prevention film 14 and the inflating agent 11, which is covered with the air shutoff film 13, are sequentially inserted from the upper side into the internal space 9 of the cylindrical casing 2 (see FIG. 3). The barrier film 12 is brought into contact with the upper surface of the coupling part 4b, while the lower surface of the air shutoff film 13 is brought into contact with the inner surface of the backflow prevention film 14.

Subsequently, the lid 3 is joined on the upper end of the cylindrical casing 2 (see FIG. 4). When the cylindrical casing 2 is joined on the lid 3, the internal space 9 is formed, and the drug 10 and the inflating agent 11 are sealed into the internal space 9.

Then, the sealing film 15 is attached to the upper surface of the lid 3 to close the air inflow holes 6a, which completes the assembly of the drug delivery device 1 (see FIG. 1).

### (The Work and the Operation During Use of the Drug Delivery Device According to the First Embodiment)

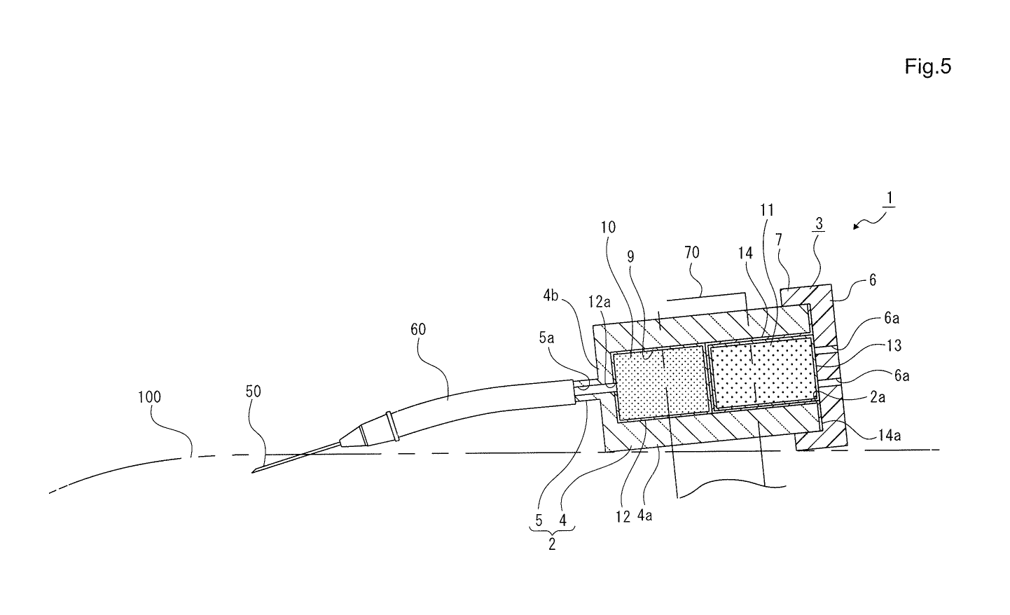

In the following, the work and the operation during use of the drug delivery device 1 will be described (see FIG. 5 through FIG. 7).

First, with the cylindrical end 5 of the cylindrical casing 2 facing upward, the cap 8 is removed from the cylindrical end 5, and the puncture jig (not shown) is inserted through the delivery hole 5a of the cylindrical end 5 to form an insertion hole 12a in the barrier film 12, and after the puncture jig is pulled out of the cylindrical end 5, a needle 50 or a tube 60 with the needle 50 joined at the tip portion is mounted on the cylindrical end 5 (see FIG. 5). It is desirable that the tube 60 is formed of a highly flexible material such as a rubber material.

Next, the needle 50 is stuck into the skin 100 at a position where the drug 10 is to be injected, and the cylindrical casing 2 is fixed on the arm or the skin 100 by means of an adhesive tape 70, a belt 70, or the like.

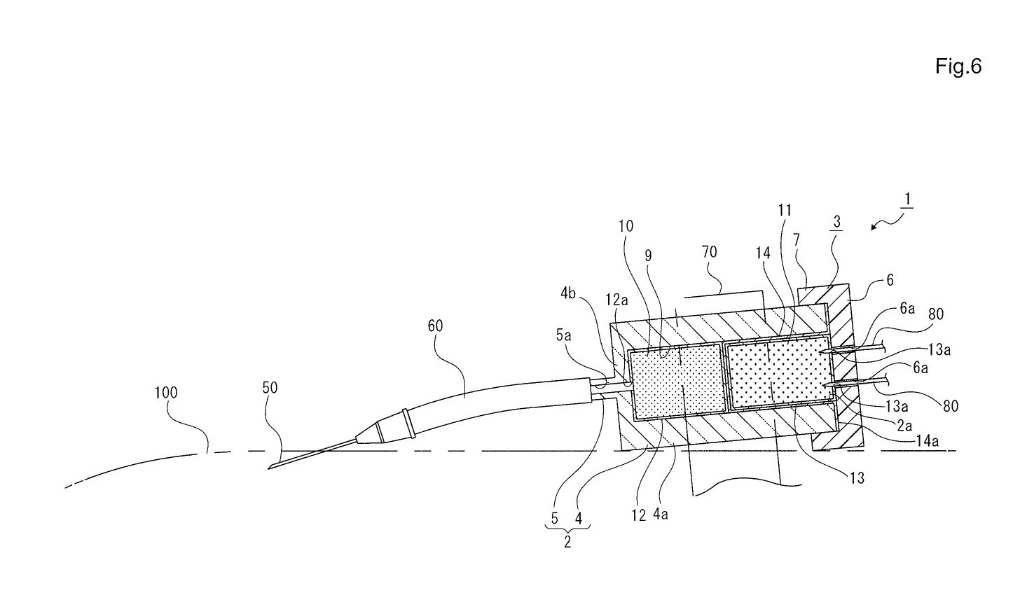

Then, the sealing film 15 is detached from the lid 3, a needle-like boring jig 80 is inserted into the air inflow hole 6a, and the tip of the boring jig 80 is pressed against the air shutoff film 13 (see FIG. 6). When the tip of the boring jig 80 is pressed against the air shutoff film 13, a through-hole 13a is formed in the air shutoff film 13.

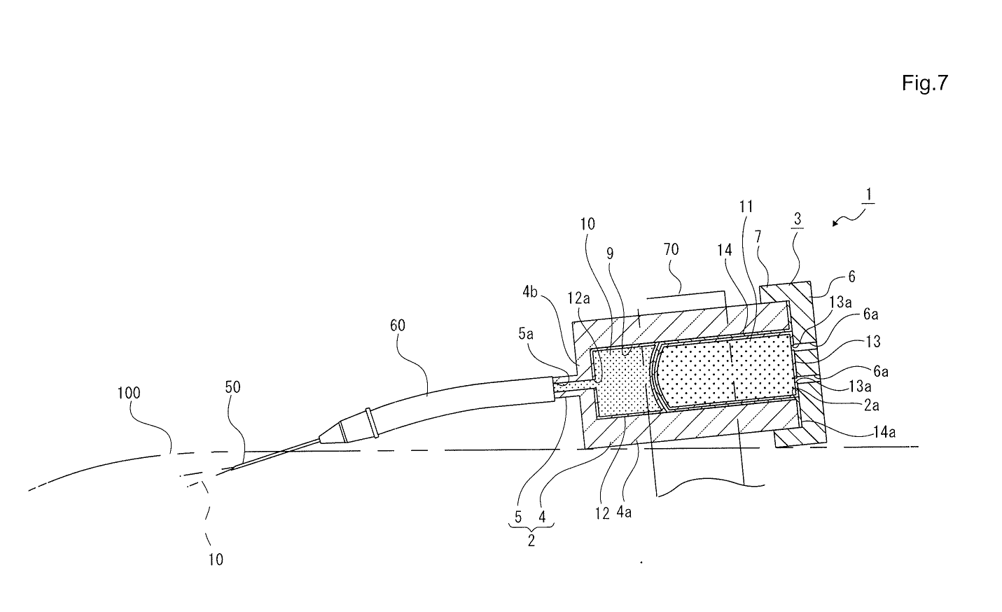

When the through-hole 13a is formed in the air shutoff film 13, air flows to the inside of the air shutoff film 13 from the through-hole 13a through the air inflow hole 6a, and as the oxygen and the moisture in the air produce iron oxide, the inflating agent 11 is inflated (see FIG. 7). As the inflating agent 11 is inflated, pressure is applied to the drug 10. Thereupon, the drug 10 is delivered into the body from the needle 50 stuck in the skin 100.

Where the inflating agent 11 is sealed in the internal space 9 by the lid 3 and the sealing film 15 and a container bag formed of a non-woven fabric, etc. is used instead of the air shutoff film 13, simply detaching the sealing film 15, without performing the work of forming the through-hole 13a in the air shutoff film 13 using the boring jig 80, leads to production of iron oxide by the oxygen and the moisture in the air, so that the inflating agent 11 is inflated.

Therefore, where the container bag is used instead of the air shutoff film 13, the work during injection of the drug 10 is simpler, and the ease of use of the drug delivery device 1 can be improved.

As described above, since in the drug delivery device 1 the drug 10 is sealed under the inflating agent 11, it is less likely that the opening on the upper side of the delivery hole 5a is closed by the air shutoff film 13, which holds the inflating agent 11, when the drug 10 is pressed from above during inflation of the inflating agent 11 and the drug 10 is sequentially sent into the delivery hole 5a from the internal space 9. Thus, the drug 10 is sent into the delivery hole 5a from the internal space 9 at a constant speed and in a constant amount, and the stable state of use of the drug delivery device 1 can be secured.

If one wants to stop the delivery of the drug 10 while the drug delivery device 1 is in use, one has only to stop the inflation of the inflating agent 11 while preventing airflow into the inside of the air shutoff film 13 by attaching the sealing film 15 back to the lid 3 to close the air inflow hole 6a.

In the above-described example, the needle 50 is mounted at the cylindrical end 5 through the tube 60 in the drawings; however, it is also possible to mount the needle 50 at the cylindrical end 5 of the cylindrical casing 2 without interposing the tube 60, and in this case, for example, it is also possible to use the drug delivery device 1 by sticking the needle 50 into the skin 100 while the cylindrical casing 2 is hung on a hanging tool, etc. (not shown).

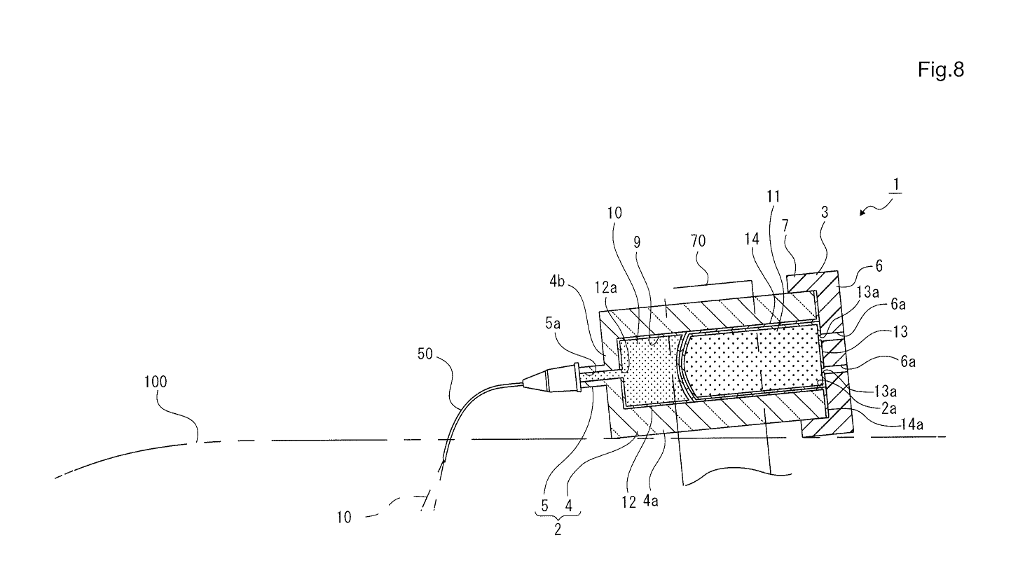

Moreover, when the needle 50 is directly mounted at the cylindrical end 5 of the cylindrical casing 2 without the interposition of the tube 60, it is possible to fix the cylindrical casing 2 on the arm or the skin 100 with the adhesive tape 70, the belt 70, or the like, for example, by using a pre-bent needle 50 (see FIG. 8).

The use of such a bent needle 50 allows the cylindrical casing 2 to be fixed on the arm or the skin 100 with the adhesive tape 70, the belt 70, or the like. Therefore, the tube 60 is no longer required and the drug delivery device 1 can be used in a simple configuration which also secures the improved ease of use.

### (Summary of the Drug Delivery Device According to the First Embodiment)

As described above, in the drug delivery device 1, the inflating agent 11 is formed of a material mainly composed of iron, and as the oxygen and the moisture in the air produce iron oxide, the inflating agent 11 is inflated and the drug 10 is delivered to the outside. Thus, the drug 10 can be reliably delivered to the outside by a simple structure.

Moreover, since the inflating agent 11 can be stored before being inflated while its volume is small, the size of the internal space 9 can be made smaller by the saved volume, and the size of the drug delivery device 1 can be reduced.

In addition, the use of the needle 50, which is to be stuck into the skin 100, or the tube 60 with the needle 50 joined at the tip portion mounted at the cylindrical end 5 allows the drug 10 to be easily injected into the body from the needle 50 by simple means, and the ease of use of the drug delivery device 1 can be improved.

### Second Embodiment

In the following, a second embodiment of the drug delivery device of the present invention will be described with reference to the accompanying drawings.

The drug delivery device according to the second embodiment is provided with multiple needle parts. In the following description, the direction in which the needle part extends is defined as the vertical direction, and the direction toward the tip of the needle part is defined as the lower side to denote the vertical direction. However, the vertical direction denoted hereinafter is intended for the purpose of illustration, and the implementation of the present invention shall not be limited by such direction.

### (The Overall Structure of the Drug Delivery Device According to the Second Embodiment)

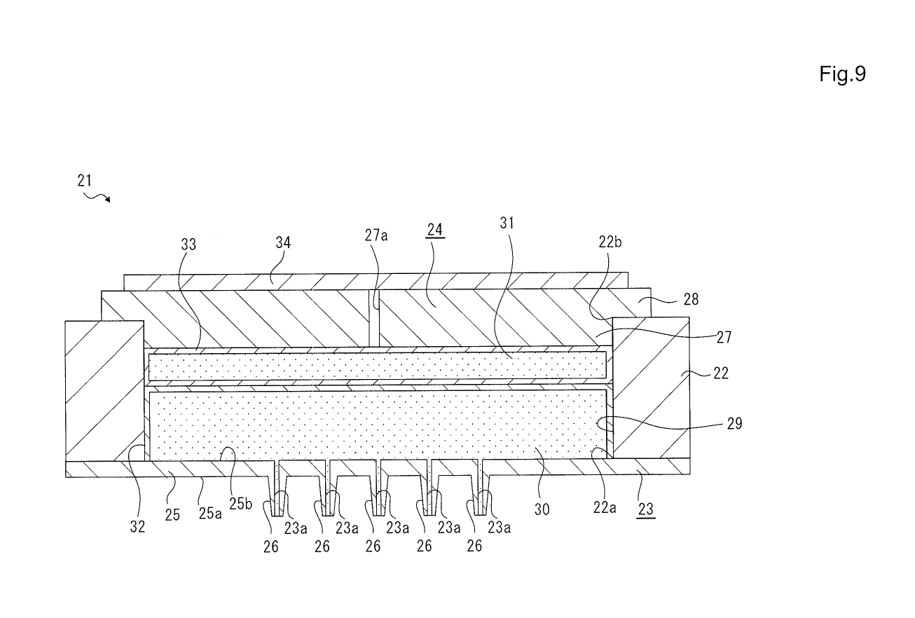

First, the overall structure of a drug delivery device 21 according to the second embodiment will be described (see FIG. 9).

The drug delivery device 21 has a cylindrical casing 22, a needle-like structure 23 and a lid 24. The cylindrical casing 22 is formed in a cylindrical shape such as a circular cylindrical shape or a rectangular cylindrical shape penetrated in the vertical direction. The needle-like structure 23 is joined on the lower end of the cylindrical casing 22. The lid 24 is joined on the upper end of the cylindrical casing 22.

The cylindrical casing 22 is formed of, for example, a highly rigid metal material such as stainless steel or a highly rigid resin material. Where the cylindrical casing 22 has an outer diameter (outer shape) of, for example, approximately 10 mm, its thickness is 0.5 mm to 2.0 mm and its height is 1 mm to 10 mm, for example. If the height of the cylindrical casing 22 is 1 mm to 10 mm, the height of the drug delivery device 21 is not excessively large, so that, when it is attached on the skin with an attaching tape to be described later, the drug delivery device 21 is less likely to be subjected to an external force of detaching the attaching tape.

The needle-like structure 23 is integrally formed of a flat base part 25, which faces in the vertical direction, and multiple needle parts 26, 26, . . . , which protrude downward from the base part 25 and are to be stuck into the skin, all made of a biocompatible material or a biodegradable polymer material. The needle-like structure 23 is formed, for example, by filling a forming die, which is formed by a photolithography process or LIGA (Lithographie Galvanoformung Abformung), with a material (a molten material or a paste material) and releasing the material after solidification from the forming die.

Examples of the biocompatible material used include polyimide and polyamide, and examples of the biodegradable polymer material used include polyactic acid, polyhydroxybutyrate, and polyethylene telephthalate. Since the needle parts 26, 26, . . . of the needle-like structure 23 are stuck into the skin, it is necessary that the needle parts have a certain rigidity so as not to be bent when they are stuck into the skin. It is therefore desirable that the needle-like structure 23 is formed of a material having an elastic modulus of 0.5 GPa or higher.

The base part 25 is formed in a disc shape or a rectangular plate-like shape, and has an outer diameter (outer shape) equal in size to the outer diameter (outer shape) of the cylindrical casing 22.

The needle parts 26, 26, . . . protrude downward from a lower surface 25a of the base part 25, and are positioned at intervals in the horizontal direction. The needle parts 26 are minute projections called microneedles, and are formed, for example, in a circular truncated conical shape or a rectangular truncated conical shape with the smaller diameter downward, and have a length of, for example, 100 μm to 200 μm, a diameter (width) of the tip of 50 μm or less, and a diameter (width) of the base end (root) of 100 μm or less.

If the diameter (width) and the length of the needle part 26 are of the above-mentioned values, the needle parts 26 are less likely to be bent when being stuck into the skin and the patient is less likely to experience pain. In addition, too small a pitch (interval) between the needle parts 26, 26, . . . makes it difficult to stick them into the skin, while too large a pitch means too small a number of the needle parts 26, 26, . . . which makes the administration of the drug to be described later insufficient. Therefore, the pitch is preferably set to a value which facilitates sticking of the needle parts into the skin and allows sufficient administration of the drug, for example, to 0.4 to 1.0 mm.

Delivery holes 23a, 23a, . . . , through each of which the drug is delivered, are formed at positions across the ends of the needle parts 26, 26, . . . from the upper surface 25b of the base part 25 of the needle-like structure 23. The delivery holes 23a are formed to penetrate the needle-like structure 23, and have a diameter of for example, 10 μm or less.

The outer circumferential portion of the base part 25 of the needle-like structure 23 is joined on the lower surface of the cylindrical casing 22. The base part 25 is joined on the cylindrical casing 22, for example, by bonding or thermocompression bonding, and when the base part 25 is joined on the cylindrical casing 22, the opening 22a on the lower side of the cylindrical casing 22 is closed by the base part 25. As an adhesive used for joining the base part 25 to the cylindrical casing 22, a biocompatible adhesive or a biodegradable polymer adhesive similar in composition to the needle-like structure 23 is used.

The lid 24 is formed of a metal material or a resin material, and is constituted of a main body 27 and a flange part 28 projecting outward from the upper end of the main body 27.

The size of the outer diameter (outer shape) of the main body 27 on the lower side of the flange part 28 is substantially equal to the size of the inner diameter (inner shape) of the needle-like structure 23. An air inflow hole 27a penetrating the main body 27 in the vertical direction is formed in the main body 27. Multiple air inflow holes 27a may be formed.

The portion of the main body 27 of the lid 24 on the lower side of the flange part 28 is fitted on the upper end of the cylindrical casing 22, and the flange part 28 is joined, for example, by means of bonding, thermocompression bonding, or the like. When the lid 24 is joined on the cylindrical casing 22, the opening 22b on the upper side of the cylindrical casing 22 is closed by the main body 27.

When the drug delivery device 21 is a type which is repeatedly usable, it is necessary to repeatedly seal a drug and an inflating agent, to be described later, into the drug delivery device 21; therefore, the lid 24 is joined on the cylindrical casing 22 in a removal state without being joined on the cylindrical casing 22 by means of bonding, thermocompression bonding, or the like.

As described above, when the needle-like structure 23 is joined on the cylindrical casing 22 from below and the lid 24 is joined on it from above, an internal space 29 is formed by the base part 25, the cylindrical casing 22, and the main body 27. The internal space 29 communicates with the delivery holes 23a, 23a, . . . of the needle-like structure 23 and the air inflow hole 27a of the lid 24.

In the internal space 29, the drug 30 and an inflating agent 31 are sealed while being separated, for example, in the vertical direction.

The drug 30 is positioned under the inflating agent 31. The drug 30 is a substance which is administered into the body through the human skin. As long as it is a water-soluble drug used as a percutaneous absorbent, there is no particular limit on the drug 30, and the drug 30 may be any drug that is administered into the body through the skin.

Examples of the drug include antipyretic analgesic, steroidal anti-inflammatory agent, vasodilating drug, antiarrhythmic agent, hypotensive drug, local anesthetic, hormonal agent, antihistamine agent, general anesthetic, hypnotic analgesic agent, antiepileptic agent, psychoneurotic agent, skeletal muscle relaxant, autonomic agent, antiparkinsonian agent, diuretic, vasoconstrictive, and respiratory stimulant.

The drug 30 is turned into a gel with low-molecular collagen or gelatin while being sealed in the internal space 29 so that, when the needle parts 26, 26, . . . are stuck into the skin, the drug is dissolved by the body heat. The drug 30 is covered with the barrier film 32 except at the surface which is in contact with the base part 25 of the needle-like structure 23. The barrier film 32 is entirely positioned between the drug 30 and the cylindrical casing 22 and between the drug 30 and the inflating agent 31, and the barrier film 32 is entirely held in close contact with the inner circumferential surface of the cylindrical casing 22 and the lower surface of the inflating agent 31.

The barrier film 32 is formed of for example, ethylene-vinyl acetate copolymer, polyethylene, polyethylene and ethylene-vinyl acetate copolymer, cyclic olefin copolymer, or polypropylene.

As described above, since the barrier film 32 is entirely positioned between the drug 30 and the cylindrical casing 22 and between the drug 30 and the inflating agent 31, the drug 30 is reliably separated from the inflating agent 31, and entry of a non-biocompatible component such as a metal ion into the drug 30 from the inflating agent 31 and the cylindrical casing 22 can be prevented, so that entry of a non-biocompatible component into the body can be prevented.

Since the surface of the drug 30 which is in contact with the base part 25 of the needle-like structure 23 is not covered with the barrier film 32, the delivery holes 23a, 23a, . . . are filled with part of the gel drug 30. The delivery holes 23a, 23a, . . . are filled with the drug 30, for example, by liquefying the drug 30 by applying heat at a temperature similar to the body temperature and pressing the drug from the internal space 29 side into the delivery holes 23a, 23a, . . . until it is sealed in to the tip of the needle parts 26, 26, . . . . When the drug 30 is sealed in the delivery holes 23a, 23a, . . . , it is desirable that the drug 30 is turned into a gel by cooling so that the drug 30 is not unnecessarily delivered from the needle parts 26, 26, . . . .

The inflating agent 31 is formed of a material mainly composed of metal powder such as iron powder, and is inflated as at least oxygen in the air produces iron oxide. More particularly, the inflating agent 31 is mainly composed of metal powder such as iron powder, and is formed of a metal halide such as salt or a reaction accelerator such as metal sulfate, water, and a water retention agent such as a polymer absorption agent.

The inflating agent 31 is sealed in the internal space 29 while being held inside the air shutoff film 33 and shut off from air. The air shutoff film 33 is formed of, for example, an ethylene-vinyl alcohol copolymer resin or a nylon-based (synthetic polymeric polyamide-based) synthetic resin.

The air shutoff film 33 is held entirely in close contact with the inner circumferential surface of the cylindrical casing 22, the lower surface of the lid 24, and the upper surface of the barrier film 32.

When a through-hole is formed in the air shutoff film 33, the air flowing in through the air inflow hole 27a of the lid 24 turns the iron into iron oxide, which causes the inflating agent 31 to generate heat and to be inflated to about 2.5 times its initial volume. As the inflating agent 31 is inflated, pressure is applied to the drug 30 by the inflation, causing the drug 30 to be delivered through the delivery holes 23a, 23a, . . . . Since the inflating agent 31 is inflated to about 2.5 times its initial volume, for example, the inflating agent 31 having a thickness of 0.5 mm can deliver the drug 30 having a thickness of 0.75 mm through the delivery holes 23a, 23a, . . . .

As described above, since the inflating agent 31 is covered with the air shutoff film 33, the inflating agent 31 is shut off from the air. It is therefore unlikely that the inflating agent 31 is accidentally inflated before delivery of the drug 30, and accidental delivery of the drug 30 can be prevented. Moreover, since the inflating agent 31 can be stored before being inflated while its volume is small, the size of the internal space 29 can be made smaller by the saved volume and the size of the drug delivery device 21 can be reduced.

A sealing film 34 is attached on the upper surface of the lid 24. As the sealing film 34, an adhesive tape, etc. which can be repeatedly attached to and detached from the lid 24 is used. The sealing film 34 seals the air inflow hole 27a of the lid 24.

When the drug delivery device 21 is used (when a drug, etc. is administered), the sealing film 34 is detached from the lid 24, and when the lid 24 is detached, the air inflow hole 27a is released from the sealed state.

With the sealing film 34 being thus attached on the lid 24, the air inflow hole 27a is sealed when the drug delivery device 21 is not in use, so that it is unlikely that a through-hole is formed in the air shutoff film 33 by a foreign substance accidentally entering the air inflow hole 27a while the drug delivery device 21 is not in use, and accidental use of the drug delivery device 21 can be prevented.

In addition, with the sealing film 34 being attached on the lid 24, it is unlikely that the airflow into the air inflow hole 27a is inhibited due to clogging caused by a foreign substance entering the air inflow hole 27a, and the drug 30 can be reliably delivered to the outside when the drug delivery device 21 is used.

In the above-described example, the cylindrical casing 22 and the needle-like structure 23 are formed as separate members; however, the needle-like structure 23 may be formed integrally with the cylindrical casing 22, and this integrally formed structure may serve as the cylindrical casing 22. In this case, the opening on the upper side of the cylindrical casing is the opening 22b, and the opening on the lower side is the opening on the lower side of the delivery hole.

### (The Assembly Procedure of the Drug Delivery Device According to the Second Embodiment)

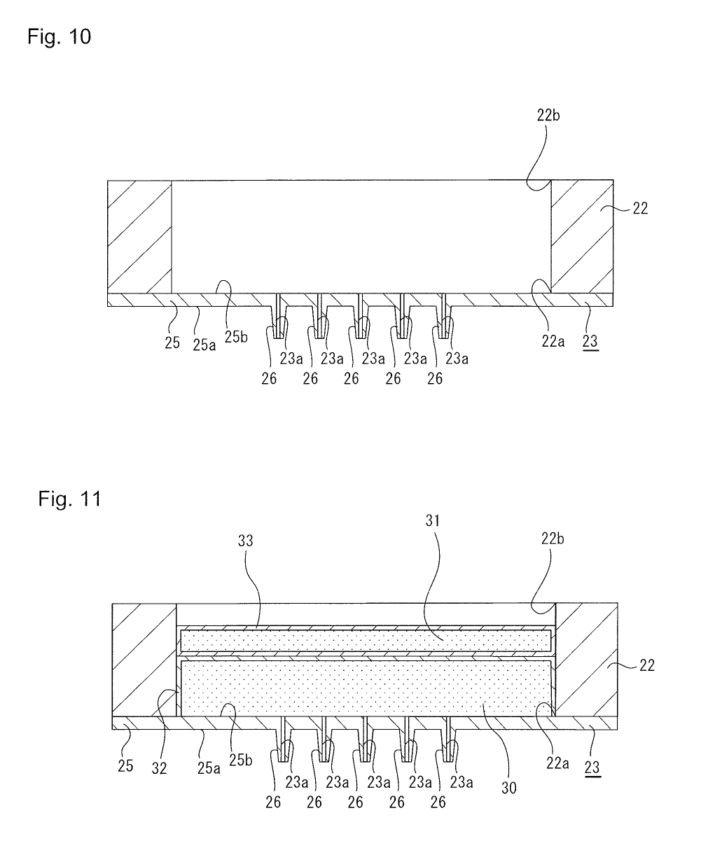

Next, the assembly procedure of the above-described drug delivery device 21 will be described (see FIG. 10 through FIG. 14).

First, the base part 25 of the needle-like structure 23 is joined on the lower end of the cylindrical casing 22 by means of bonding, thermocompression bonding, or the like (see FIG. 10).

Next, the drug 30 covered with the barrier film 32 and the inflating agent 31 held inside the air shutoff film 33 are inserted sequentially from the upper side into the cylindrical casing 22 (see FIG. 11). The lower surface of the drug 30 is brought into contact with the upper surface 25b of the base part 25, and the lower surface of the air shutoff film 33 is brought into contact with the upper surface of the barrier film 32.

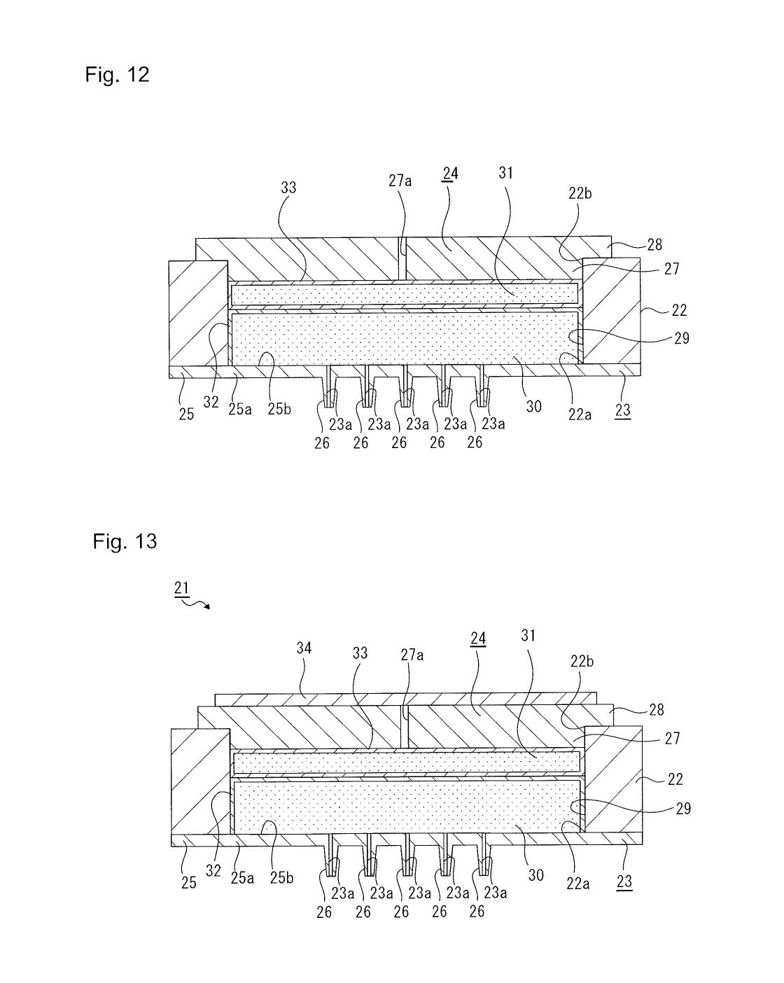

Subsequently, the lid 24 is joined on the upper end of the cylindrical casing 22 (see FIG. 12). When the cylindrical casing 22 is joined on the lid 24, the internal space 29 is formed, and the drug 30 and the inflating agent 31 are sealed into the internal space 29.

Then, the sealing film 34 is attached to the upper surface of the lid 24 to close the air inflow hole 27a, which completes the assembly of the drug delivery device 21 (see FIG. 13).

When the drug delivery device 21 is thus assembled, as described above, the drug 30 is liquefied by applying heat at the same temperature as the body temperature, and the drug 30 is pressed from the internal space 29 side into the delivery holes 23a, 23a, . . . , and then the drug 30 is turned into a gel by cooling.

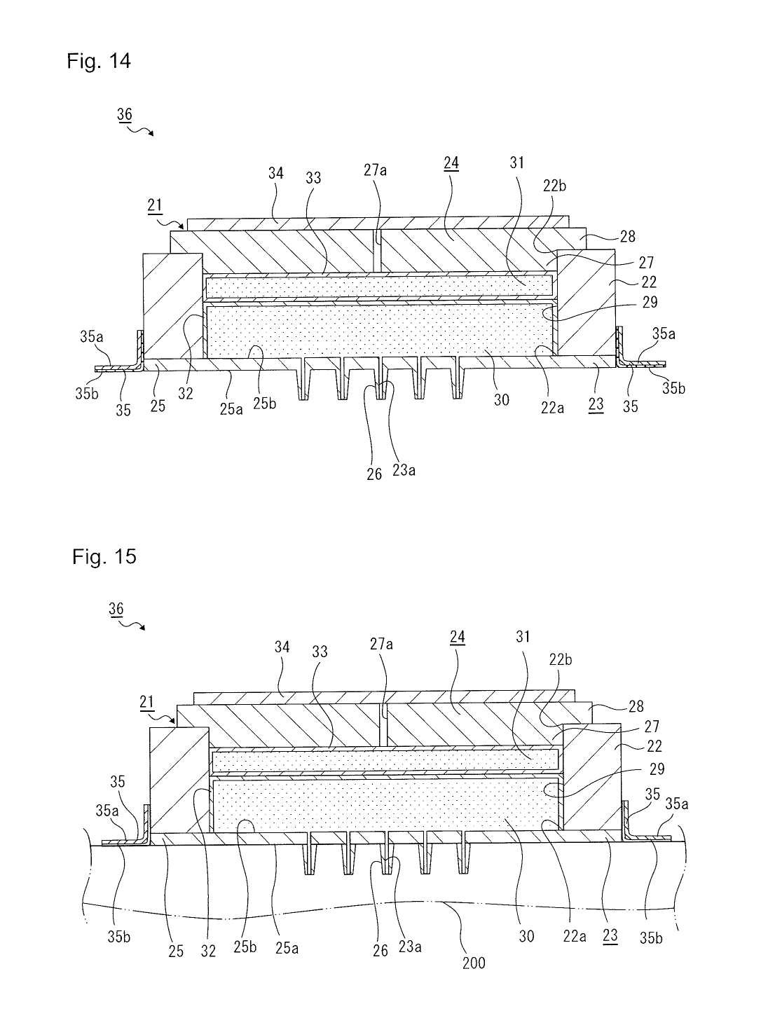

Finally, attaching tapes 35 and 35 are attached on a part of the drug delivery device 21 (see FIG. 14). When the attaching tapes 35 and 35 are attached on the part of the drug delivery device 21, a syringe 36 is formed. The attaching tapes 35 and 35 respectively have base material layers 35a and 35a and adhesive layers 35b and 35b. When the drug delivery device 21 is used, the adhesive layers 35b and 35b adhere to the skin and the adhesive tape 35 and 35 are attached.

### (The Work and the Operation During Use of the Drug Delivery Device According to the Second Embodiment)

In the following, the work and the operation during use of the drug delivery device 21 will be described (see FIG. 15 through FIG. 17).

When the drug delivery device 21 is used, as described above, the attaching tapes 35 and 35 are attached and the syringe 36 is formed.

First, the adhesive tapes 35 and 35 are attached to the skin 200, and the needle parts 26, 26, . . . of the needle-like structure 23 are stuck into the skin 200 (see FIG. 15).

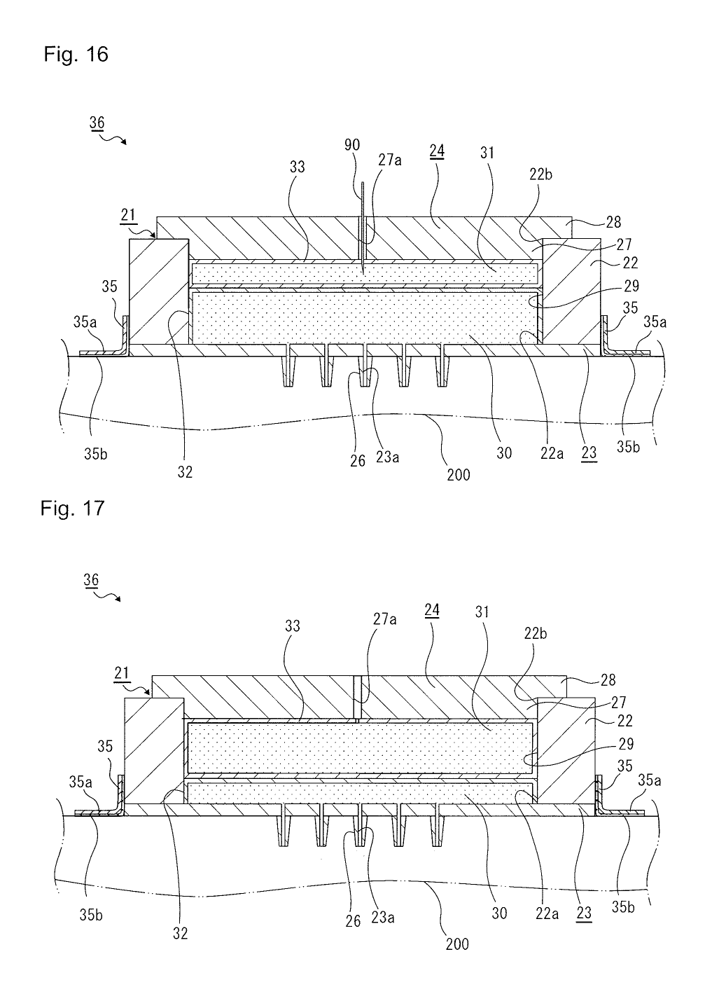

Next, the sealing film 34 is detached from the lid 24, and a needle-like boring jig 90 is inserted from the upper side of the air inflow hole 27a and the tip of the boring jig 90 is pressed against the air shutoff film 33 (see FIG. 16). When the tip of the boring jig 90 is pressed against the air shutoff film 33, a through-hole is formed in the air shutoff film 33.

When the through-hole is formed in the air shutoff film 33, air flows into the air shutoff film 33 from the through-hole through the air inflow hole 27a, and iron oxide is produced by at least oxygen contained in the air and the inflating agent 31 is inflated (see FIG. 17). When the inflating agent 31 is inflated, pressure is applied to the drug 30. At the same time, heat due to the body temperature is applied to the needle parts 26, 26, . . . stuck in the skin 200. This causes the drug 30 sealed in the delivery holes 23a, 23a, . . . to be dissolved and liquefied, and the drug 30 is delivered through the delivery holes 23a, 23a, . . . . The drug 30 delivered from the delivery holes 23a, 23a, . . . are administered through the skin 200 into the body.

As described above, since in the drug delivery device 21 the drug 30 is sealed under the inflating agent 31, it is less likely that the opening on the upper side of the delivery holes 23a, 23a, . . . are closed by the air shutoff film 33, which holds the inflating agent 31, when the drug 30 is pressed from above during inflation of the inflating agent 31 and the drug 30 is sequentially sent into the delivery holes 23a, 23a, . . . from the internal space 29. Thus, the drug 30 is sent into the delivery holes 23a, 23a, . . . from the internal space 29 at a constant speed and in a constant amount, and the stable state of use of the drug delivery device 21 can be secured.

If one wants to stop the delivery of the drug 30 while the drug delivery device 21 is in use, one has only to stop the inflation of the inflating agent 31 while preventing airflow into the inside of the air shutoff film 33 by attaching the sealing film 34 back to the lid 24 to close the air inflow hole 27a.

As described above, in the drug delivery device 21, the inflating agent 31 is formed of a material mainly composed of iron, and as at least the oxygen in the air produces iron oxide, the inflating agent 31 is inflated and the drug 30 is delivered to the outside. Thus, the drug 30 can be reliably delivered to the outside by a simple structure. Moreover, since the inflating agent 31 can be stored before being inflated while its volume is small, the size of the internal space 29 can be made smaller by the saved volume, and the size of the drug delivery device 21 can be reduced.

### (Modified Examples of the Drug Delivery Device According to the Second Embodiment)

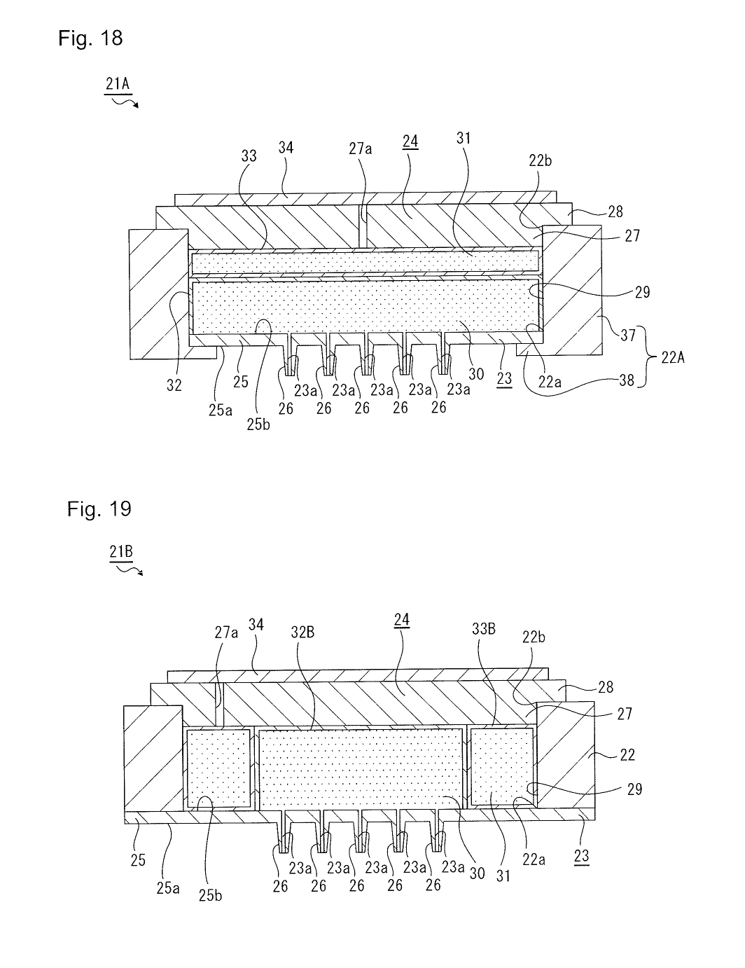

In the following, modified examples of the drug delivery device will be described (see FIG. 18 and FIG. 19).

First, a drug delivery device 21A according to a first modified example will be described (see FIG. 18). The drug delivery device 21A according to the first modified example to be described below is different from the above-described drug delivery device 21 only in the shape of the cylindrical casing; therefore, only the difference part from the drug delivery device 21 will be described in detail, while the other parts will be denoted by the same reference signs as given to the same parts in the drug delivery device 21 and the description thereof will be omitted.

The drug delivery device 21A has a cylindrical casing 22A, and the cylindrical casing 22A is constituted of a cylindrical part 37 which is formed in a cylindrical shape such as a circular cylindrical shape or a rectangular cylindrical shape penetrated in the vertical direction, and an inner flange part 38 projecting inward from the lower end of the cylindrical part 37.

The base part 25 of the needle-like structure 23 has the outer diameter (outer shape) equal in size to the inner diameter (inner shape) of the cylindrical part 37. The outer circumferential portion of the base part 25 of the needle-like structure 23 is joined on the upper surface of the inner flange part 38.

In the drug delivery device 21A, since the inner flange part 38 is provided, it is possible to assemble the drug delivery device 21A by sequentially inserting the needle-like structure 23, the drug 30 covered with the barrier film 32, and the inflating agent 31 held inside the air shutoff film 33 from the upper side into the cylindrical casing 22.

Thus, the drug delivery device 21A is easy to assemble, and it can reduce the manufacturing cost and shorten the manufacturing time.

Next, a drug delivery device 21B according to a second modified example will be described (see FIG. 19). The drug delivery device 21B according to the second modified example to be described below is different from the above-described drug delivery device 21 only in the sealed state of the drug and the inflating agent in the internal space; therefore, only the difference part from the drug delivery device 21 will be described in detail, while the other parts will be denoted by the same reference signs as given to the same parts in the drug delivery device 21 and the description thereof will be omitted.

In the drug delivery device 21B, the drug 30 and the inflating agent 31 are sealed in the internal space 29 while being separated, for example, on the inner side and the outer side.

The drug 30 has a circular columnar shape or a rectangular columnar shape and is sealed in a part at the center of the internal space 29 and is covered with a barrier film 32B except at the surface which is in contact with the base part 25 of the needle-like structure 23. The surface of the drug 30 which is in contact with the base part 25 of the needle-like structure 23 is positioned on the upper side of the delivery holes 23a, 23a, . . . formed in the needle-like structure 23.

The inflating agent 31 has an annular shape, is sealed in a part on the outer circumferential side of the internal space 29 while being held inside an air shutoff film 33B, and is positioned on the outer circumferential side of the drug 30. The air shutoff film 33B is held entirely in close contact with the inner circumferential surface of the cylindrical casing 22, the lower surface of the lid 24, the upper surface 25b of the base part 25, and the outer circumferential surface of the drug 30.

The air inflow hole 27a is formed in the lid 24 at a position where it communicates with the air shutoff film 33B.

The drug delivery device 21B shows one example of the sealed state of the drug 30 and the inflating agent 31 in the internal space 29; as long as the positional relation between the drug 30 and the inflating agent 31 is such that the inflation of the inflating agent 31 causes the drug 30 to be delivered through the delivery holes 23a, 23a, . . . , the drug 30 and the inflating agent 31 can be held in any sealed state in the internal space 29.

### (Summary of the Drug Delivery Device According to the Second Embodiment)

As has been described, in the drug delivery devices 21, 21A, and 21B, the internal space 29, which communicates with the delivery holes 23a, 23a, . . . and the air inflow hole 27a and in which the inflating agent 31 and the drug 30 are sealed while being separated from each other, is formed by the base part 25 of the needle-like structure 23, the cylindrical casing 22, and the lid 24, and the drug delivery devices are configured such that inflation of the inflating agent 31 causes the drug 30 to be delivered through the delivery holes 23a, 23a, . . . .

Accordingly, the drug 30 sealed in the internal space 29 is delivered through the delivery holes 23a, 23a, . . . as the inflating agent 31 is inflated while the multiple needle parts 26, 26, . . . are stuck into the skin of a patient. Thus, the need for structures such as a reservoir, an electric circuit, and a pump, is eliminated, and simplification of the structure as well as reduction of the size of the device can be realized.

What is claimed is:

1. A drug delivery device comprising:
a cylindrical casing formed in a shape which is at least partly cylindrical and has two openings on opposite sides; and
a lid which has an air inflow hole and is joined at one end of the cylindrical casing so as to close one of the openings, wherein
an internal space, in which an inflating agent and a drug are sealed while being separated from each other, is formed by at least the cylindrical casing and the lid,
the internal space communicates with the air inflow hole and a delivery hole through which the drug is delivered,
the inflating agent is formed of a material which is mainly composed of iron,
upon inflow of air through the air inflow hole, iron oxide is produced by at least oxygen in the inflow air, which causes the inflating agent to be inflated, and as the inflating agent is inflated, pressure is applied to the drug and the drug is delivered through the delivery hole.

2. The drug delivery device according to claim 1, wherein a sealing film, which closes the air inflow hole, is attached on the lid to stop the delivery of the drug through the delivery hole.

3. The drug delivery device according to claim 1, wherein
the drug is sealed in the internal space while being covered with a barrier film, and
the barrier film is entirely positioned between the drug and the cylindrical casing and between the drug and the inflating agent.

4. The drug delivery device according to claim 2, wherein
the drug is sealed in the internal space while being covered with a barrier film, and
the barrier film is entirely positioned between the drug and the cylindrical casing and between the drug and the inflating agent.

5. The drug delivery device according to claim 1, wherein a backflow prevention film, which prevents inflow of the drug to the inflating agent, is disposed at least between the inflating agent and the drug in the internal space.

6. The drug delivery device according to claim 2, wherein a backflow prevention film, which prevents inflow of the drug to the inflating agent, is disposed at least between the inflating agent and the drug in the internal space.

7. The drug delivery device according to claim 3, wherein a backflow prevention film, which prevents inflow of the drug to the inflating agent, is disposed at least between the inflating agent and the drug in the internal space.

8. The drug delivery device according to claim 4, wherein a backflow prevention film, which prevents inflow of the drug to the inflating agent, is disposed at least between the inflating agent and the drug in the internal space.

9. The drug delivery device according to claim 1, wherein
the cylindrical casing is provided with a cylindrical end having the delivery hole, and
a needle which is stuck into skin or a tube with the needle joined at a tip portion is mounted at the cylindrical end.

10. The drug delivery device according to claim 2, wherein
the cylindrical casing is provided with a cylindrical end having the delivery hole, and
a needle which is stuck into skin or a tube with the needle joined at a tip portion is mounted at the cylindrical end.

11. The drug delivery device according to claim 3, wherein
the cylindrical casing is provided with a cylindrical end having the delivery hole, and
a needle which is stuck into skin or a tube with the needle joined at a tip portion is mounted at the cylindrical end.

12. The drug delivery device according to claim 4, wherein
the cylindrical casing is provided with a cylindrical end having the delivery hole, and
a needle which is stuck into skin or a tube with the needle joined at a tip portion is mounted at the cylindrical end.

13. The drug delivery device according to claim 5, wherein
the cylindrical casing is provided with a cylindrical end having the delivery hole, and
a needle which is stuck into skin or a tube with the needle joined at a tip portion is mounted at the cylindrical end.

14. The drug delivery device according to claim 6, wherein
the cylindrical casing is provided with a cylindrical end having the delivery hole, and
a needle which is stuck into skin or a tube with the needle joined at a tip portion is mounted at the cylindrical end.

15. The drug delivery device according to claim 7, wherein
the cylindrical casing is provided with a cylindrical end having the delivery hole, and
a needle which is stuck into skin or a tube with the needle joined at a tip portion is mounted at the cylindrical end.

16. The drug delivery device according to claim 8, wherein
the cylindrical casing is provided with a cylindrical end having the delivery hole, and
a needle which is stuck into skin or a tube with the needle joined at a tip portion is mounted at the cylindrical end.
